(19) United States
(12) Patent Application Publication
(10) Pub. No.: US 2023/0255938 A1
(43) Pub. Date: Aug. 17, 2023

(54) COMPOSITION FOR INHIBITING SEBUM SECRETION COMPRISING FABP4 INHIBITOR

(71) Applicant: THE INDUSTRY & ACADEMIC COOPERATION IN CHUNGNAM NATIONAL UNIVERSITY (IAC), Daejeon (KR)

(72) Inventors: Chong Won CHOI, Yongin-si (KR); Chang Deok KIM, Daejeon (KR); Jung-Min SHIN, Daejeon (KR); Jin Gwi YOO, Ansan-si (KR)

(73) Assignee: THE INDUSTRY & ACADEMIC COOPERATION IN CHUNGNAM NATIONAL UNIVERSITY (IAC), Daejeon (KR)

(21) Appl. No.: 18/168,701

(22) Filed: Feb. 14, 2023

(30) Foreign Application Priority Data
Feb. 16, 2022 (KR) 10-2022-0020219

(51) Int. Cl. A61K 31/415; A61K 8/49; A61Q 19/00; A61P 17/00

(52) U.S. Cl. CPC A61K 31/415; A61K 8/494; A61Q 19/008; A61P 17/00

(57) ABSTRACT

The present invention relates to a composition for inhibiting sebum secretion comprising a FABP4 inhibitor, more specifically, relates to a composition for inhibiting sebum secretion capable of regulating sebum secretion by reducing lipid synthesis in sebocytes, inhibiting lipid synthesis-related signal transduction systems, and reducing the expression of lipid synthesis-related electronic factors. Also, the composition of the present invention can be used for preventing, treating or improving acne or seborrheic dermatitis.

### BACKGROUND

### Technical Field

The present invention relates to a composition for inhibiting sebum secretion comprising a FABP4 inhibitor.

### Background Art

The sebaceous gland is a tissue of epidermal origin and is located around the hair follicle. Sebum is made from sebocytes that make up sebaceous glands and is discharged to the skin surface through pores, and sebum is close to the aqueous phase like water and forms a sebum film on the skin surface to prevent skin from drying out. In addition, sebum plays an important role in maintaining skin homeostasis, such as controlling skin temperature, inhibiting the invasion of external microorganisms, and maintaining skin in a slightly acidic state.

Sebocytes produce sebum through fat synthesis. It is known that the process of biosynthesis of fat by sebocytes is regulated by various cytokines and vitamins in addition to hormones. Sebocytes synthesize sebum through the expression of various transcription factors and enzymes involved in fat synthesis in sebocytes under the control of these various substances.

Proper sebum secretion is important for healthy skin. It is well known that various skin diseases occur when sebum, which functions to maintain skin homeostasis, is excessively secreted. In particular, the face is an area where sebum is secreted more than other skin areas, and when sebum is excessively secreted, pores widen and acne is caused or worsened. In addition, excessive sebum secretion exacerbates seborrheic dermatitis, causes discomfort by making the makeup smudged, or the skin greasy.

There is a lack of effective and safe methods to control excessive sebum secretion. Currently, the most representative method for regulating sebum synthesis in sebocytes is a method using isotretinoin, a vitamin A derivative. Isotretinoin acts on sebocytes and effectively reduces sebum synthesis, but it is difficult to use isotretinoin for a long time due to side effects such as lipid metabolism disorders such as blood triglycerides and cholesterol, and liver toxicity that occur when taking isotretinoin. In addition, isotretinoin causes teratogenicity in women of childbearing age, so it is difficult to use in young women who are mainly affected by acne. Therefore, research is being conducted to find a substance that effectively reduces the sebum synthesis with fewer side effects, but a substance that can replace isotretinoin has not yet been found.

FABP (fatty acid binding protein) is a 14-15 kDa protein present in cells and plays an important role in fat metabolism, such as fat synthesis. It is known that FABP binds to hydrophobic ligands such as saturated/unsaturated fatty acids, eicosanoids, and endocannabinoids to store these substances in cells or and acts as a chaperone in the process of transporting these substances to intracellular organelles such as mitochondria, endoplasmic reticulum, and nucleus. There are various subtypes of FABP in the human body. To date, 9 types of FABP subtypes have been discovered, and new subtypes are continuously being discovered. Among these subtypes, it is known that FABP4 (adipocyte FABP) is expressed in adipocytes that synthesize fat, and FABPS (epidermal FABP) is expressed in epidermal cells.

FABP4 was first discovered in adipocytes. Its expression is regulated according to the differentiation of adipocytes, and it is known to be regulated by fatty acids, PPAR-γ agonists and insulin. In the case of adipocytes obtained from FABP4-deficient mice, it is known that lipolysis is reduced, suggesting a role in fat metabolism. FABP4 is also expressed in monocytes/macrophages, and it has been found that the expression of inflammatory cytokines, such as TNF-α, IL-1β, IL-6, and MCP-1, is suppressed in FABP4-deficient monocytes/macrophages, suggesting that FABP4 is also involved in the inflammatory response.

Various attempts have been made to regulate fat metabolism and inflammatory responses using chemical inhibitors of FABP4. To date, various FABP4 inhibitors have been synthesized, and it has been confirmed that insulin resistance, arteriosclerosis, and metabolic diseases are reduced in animal experiments when administered.

Korean Patent Publication No. 2021-0098732 discloses a composition for promoting cilia formation comprising a novel FABP4 inhibitor as an active ingredient, and Korean Patent No. 1841350 relates to a non-viral gene transfer complex targeting adipocytes containing a double plasmid vector and discloses the use of a dual plasmid vector capable of inhibiting FABP4 and FABP5 for the treatment of obesity and obesity-induced metabolic syndrome.

However, it has not been reported that a specific FABP4 inhibitor inhibits sebum secretion as in the present invention.

### DETAILED DESCRIPTION OF THE INVENTION

### Technical Problem

An object of the present invention is to provide a composition for inhibiting sebum secretion comprising a FABP4 inhibitor. More specifically, the present invention is to provide a composition for preventing or treating acne or seborrheic dermatitis comprising a FABP4 inhibitor.

### Technical Solution

In order to achieve the object, the present invention provides a composition for inhibiting sebum secretion comprising a compound represented by Formula 1 as an active ingredient.

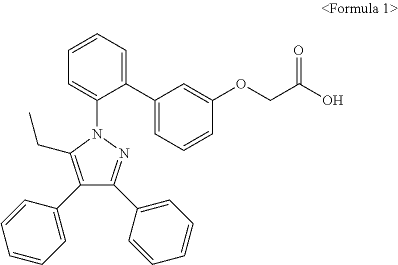 [SMILES: CCc1c(-c2ccccc2)c(-c2ccccc2)nn1-c1ccccc1-c1cccc(OCC(=O)O)c1]

In one embodiment of the present invention, the present invention provides a pharmaceutical composition for preventing or treating diseases caused by sebum secretion, comprising a compound represented by Formula 1 as an active ingredient.

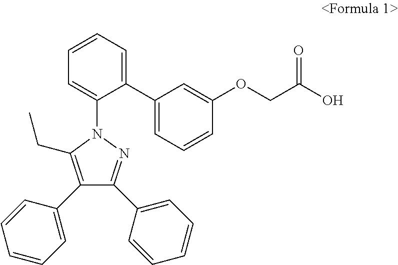 [SMILES: CCc1c(-c2ccccc2)c(-c2ccccc2)nn1-c1ccccc1-c1cccc(OCC(=O)O)c1]

The diseases caused by the sebum secretion include all diseases caused by sebum secretion, and are preferably one or more selected from the group consisting of acne, rosacea, seborrheic eczema, seborrheic dermatitis, and seborrheic alopecia, but are not limited thereto.

The pharmaceutical composition may further comprise one or more components effective for diseases caused by sebum secretion, wherein the components may comprise all components known in the art to which the present invention belongs.

The pharmaceutical composition is preferably a formulation selected from the group consisting of ointments, powders, tablets, capsules, powders, solutions, gels, pastes, patches and granules, but is not limited thereto.

When formulating the pharmaceutical composition of the present invention, it may be prepared using diluents or excipients such as commonly used fillers, extenders, binders, wetting agents, disintegrants, and surfactants. Also, sterilized aqueous solutions, non-aqueous solvents, suspensions, emulsions, freeze-dried preparations, and suppositories are included. Propylene glycol, polyethylene glycol, vegetable oils such as olive oil, and injectable esters such as ethyl oleate may be used as non-aqueous solvents and suspending agents.

In another example of the present invention, the present invention provides a cosmetic composition for preventing or improving skin conditions caused by sebum secretion, comprising a compound represented by Formula 1 as an active ingredient.

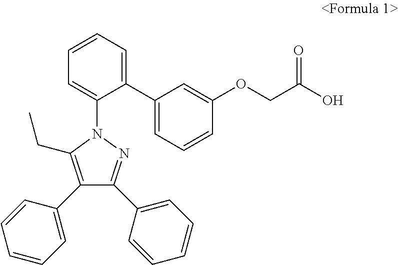 [SMILES: CCc1c(-c2ccccc2)c(-c2ccccc2)nn1-c1ccccc1-c1cccc(OCC(=O)O)c1]

The skin condition due to sebum secretion includes all skin conditions caused by sebum secretion, and is preferably one or more selected from the group consisting of smudged makeup, glossiness, skin trouble, and scab, but is not limited thereto.

The cosmetic composition may further comprise one or more components effective for skin conditions caused by sebum secretion, and the components may include all components known in the art to which the present invention belongs.

The cosmetic composition may be selected from the group consisting of solutions, suspensions, emulsions, pastes, gels, creams, powders, soaps, cleansing containing surfactant, oils and sprays.

The cosmetics comprise antioxidants, stabilizers, solubilizers, vitamins, conventional adjuvants such as pigments and fragrances, and carriers.

In the case of a paste, cream or gel, animal oil, vegetable oil, wax, paraffin, starch, tragacantha, cellulose derivative, polyethylene glycol, silicone, bentonite, silica, talc or zinc oxide may be used as a carrier component.

When the formulation of the present invention is a powder or spray, lactose, talc, silica, aluminum hydroxide, calcium silicate or polyamide powder may be used as a carrier component, and in particular, in the case of a spray, a propellant such as chlorofluorohydrocarbon, propane/butane or dimethyl ether may be additionally comprised.

When the formulation of the present invention is a solution or emulsion, a solvent, solubilizing agent or emulsifying agent is used as a carrier component, such as water, ethanol, isopropanol, ethyl carbonate, ethyl acetate, benzyl alcohol, benzyl benzoate, propylene glycol, 1 ,3-butyl glycol oil, glycerol aliphatic esters, polyethylene glycol or fatty acid esters of sorbitan.

When the formulation of the present invention is a suspension, as a carrier component, a liquid diluent such as water, ethanol or propylene glycol, a suspending agent such as ethoxylated isostearyl alcohol, polyoxyethylene sorbitol ester and polyoxyethylene sorbitan ester, microcrystalline cellulose, aluminum metahydroxide, bentonite, agar or tragacantha and the like may be used.

When the formulation of the present invention is a surfactant-containing cleansing, as a carrier component, aliphatic alcohol sulfate, aliphatic alcohol ether sulfate, sulfosuccinic acid monoester, isethionate, imidazolinium derivative, methyl taurate, sarcosinate, fatty acid amide ether sulfate, alkylamidobetaine, aliphatic alcohol, fatty acid glycerides, fatty acid diethanolamides, vegetable oils, lanolin derivatives, or ethoxylated glycerol fatty acid esters may be used.

In another embodiment of the present invention, the present invention provides a method of inhibiting sebum secretion in a non-human animal, comprising a step of administering a compound represented by Formula 1.

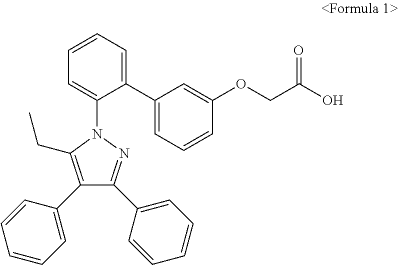 [SMILES: CCc1c(-c2ccccc2)c(-c2ccccc2)nn1-c1ccccc1-c1cccc(OCC(=O)O)c1]

### Effects of the invention

The present invention provides a composition for inhibiting sebum secretion comprising a FABP4 inhibitor capable of controlling sebum secretion by reducing lipid synthesis in sebocytes, inhibiting lipid synthesis-related signal transduction systems, and reducing the expression of lipid synthesis-related electronic factors. More specifically, FABP4 inhibitor of the present invention can be used as a composition for reducing sebum, and for improving seborrheic dermatitis and acne.

### BRIEF DESCRIPTION OF THE DRAWINGS

FIG. 1 shows changes in the expression of differentiation-related factors of sebocytes and changes in adipogenesis of sebocytes when FABP4 inhibitor was treated in sebocytes.

### DETAILED DESCRIPTION

Hereinafter, examples are presented to aid understanding of the present invention.

However, the following examples are provided to more easily understand the present invention, and the content of the present invention is not limited by the following examples.

### EXAMPLE 1 CELL CULTURE

Immortalized human sebocytes were used for the experiment. The cell lines were established as previously described. All procedures were approved by the Institutional Committee of Chungnam National University Hospital. The immortalized human sebocytes were cultured in Sebomed medium (Biochrom, Berlin, Germany) supplemented with 10% fetal bovine serum (Gibco BRL, Rockville, Md., USA) and 5 ng/mL of recombinant human EGF (Invitrogen, Grand Island, N.Y., USA).

### EXAMPLE 2 FABP4 INHIBITOR

The FABP4 inhibitor of the present invention is a compound of Formula 1.

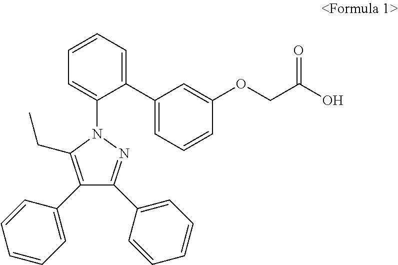 [SMILES: CCc1c(-c2ccccc2)c(-c2ccccc2)nn1-c1ccccc1-c1cccc(OCC(=O)O)c1]

### EXAMPLE 3 WESTERN BLOTTING

After harvesting, the cells were lysed in protein extraction solution (Intron, Daejeon, Korea). Equal amounts of protein were then loaded and separated by SDA-PAGE, and then the proteins were transferred onto nitrocellulose membranes (Pall Corp., Port Washington, N.Y., USA). After blocking with 5% skim milk, the membranes were incubated with various primary antibodies. The blot was reacted with a secondary antibody conjugated with peroxidase, and were visualized for specific proteins with ECL (BIOMAX, Seoul, Korea). The primary antibodies used in western blot analysis were as follows: Actin, SREBP-1(Santa Cruz, Calif.), PPAR-γ, phospho-IGFR (p-IGFR), phospho-Akt (Cell Signaling Technology, Danvers, Mass.)

### EXAMPLE 4 THIN-LAYER CHROMATOGRAPH, TLC

For quantitative analysis of intracellular lipids, the present invention used the TLC method as used in previous studies. Briefly, immortalized human sebocytes were cultured in a medium containing 2 μCi of ¹⁴C-acetate and sodium salt (PerkinElmer, Boston, Mass., USA) and allowed to react for 4 hours. Intracellular lipids were extracted with chloroform and methanol (2:1, v/v). The solvent was evaporated and the lipid was resuspended in chloroform. TLC (TLC silica gel 60 F₂₅₄, Merck KgaA) was used to separate intracellular lipids. After development with hexane and ethyl acetate (6:1, v/v), intracellular lipids were visualized by radioactivity measurement.

### Test Example

The effect of BMS309403 on IGF-1-induced lipid synthesis was measured using an immortalized human sebaceous cell line established in a previous study. Immortalized human sebocytes were treated with 1, 2 or 5 μM of BMS309403. After treatment with BMS309403 for 1 hour, sebocytes were treated with 50 ng/mL of IGF-1 to induce differentiation and lipid synthesis.

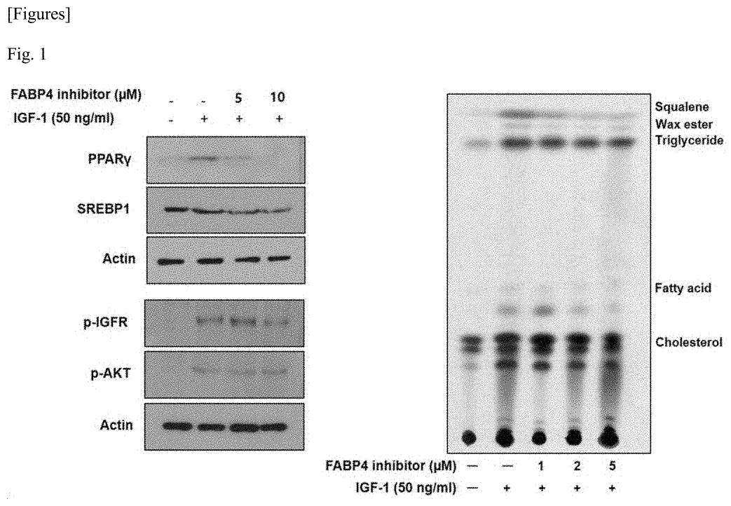

To investigate the effect of BMS309403 on IGF-1-induced lipid synthesis in immortalized human sebocytes, transcription factors and enzymes related to sebocytes differentiation and lipid synthesis were investigated. In the group in which immortalized human sebocytes were treated with IGF-1, the levels of various transcription factors related to sebocyte differentiation, such as SREBP-1 and PPAR-γ were increased, but BMS309403 decreased their levels in a dose-dependent manner (see upper left of FIG. 1).

As the present inventors previously reported the role of the Akt/mTOR signaling pathway in sebocytes in IGF-1-induced fat synthesis, we examined whether BMS309403 regulates the Akt signaling pathway. In the present invention, IGF-1 increased the phosphorylation of IGFR and Akt, a downstream effector. On the contrary, BMS309403 markedly reduced phosphorylation of IGFR and Akt in a dose-dependent manner (see lower left of FIG. 1).

Through the results of TLC performed 48 hours after IGF-1 treatment, it was confirmed that IGF-1 treatment increased lipid accumulation in immortalized human sebocytes, and BMS309403 reduced intracellular lipid accumulation in a dose-dependent manner. IGF-1 increased the Intracellular lipid synthesis such as cholesterol, triglycerides, wax esters and squalene in immortalized human sebocytes. However, BMS309403 treatment reduced IGF-1-induced lipid synthesis. Among various lipids, the synthesis of squalene and wax esters was significantly reduced by BMS309403 (see the right side of FIG. 1).

What is claimed:

1. A composition for inhibiting sebum secretion comprising a compound represented by Formula 1 as an active ingredient

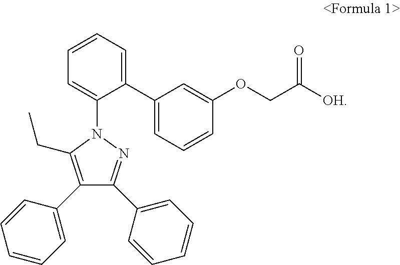 [SMILES: CCc1c(-c2ccccc2)c(-c2ccccc2)nn1-c1ccccc1-c1cccc(OCC(=O)O)c1]

2. A pharmaceutical composition for preventing or treating diseases caused by sebum secretion, comprising a compound represented by Formula 1 as an active ingredient

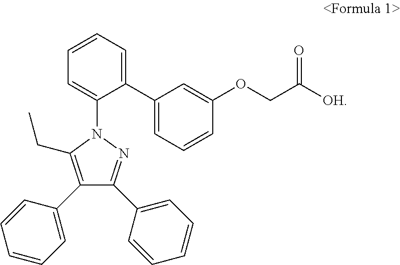 [SMILES: CCc1c(-c2ccccc2)c(-c2ccccc2)nn1-c1ccccc1-c1cccc(OCC(=O)O)c1]

3. The pharmaceutical composition according to claim 2, wherein the diseases caused by sebum secretion are preferably one or more selected from the group consisting of acne, rosacea, seborrheic eczema, seborrheic dermatitis, and seborrheic alopecia.

4. The pharmaceutical composition according to claim 2, further comprising one or more components effective for diseases caused by sebum secretion.

5. The pharmaceutical composition according to claim 2, wherein the composition has a formulation selected from the group consisting of ointments, powders, tablets, capsules, powders, solutions, gels, pastes, patches and granules.

6. A cosmetic composition for preventing or improving skin conditions caused by sebum secretion, comprising a compound represented by Formula 1 as an active ingredient

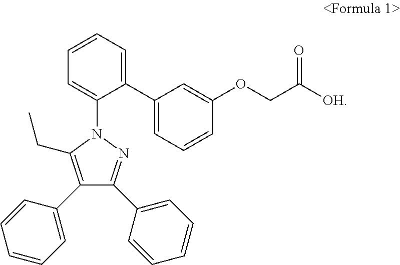 [SMILES: CCc1c(-c2ccccc2)c(-c2ccccc2)nn1-c1ccccc1-c1cccc(OCC(=O)O)c1]

7. The cosmetic composition according to claim 6, wherein the skin conditions caused by sebum secretion are one or more selected from the group consisting of smudged makeup, glossiness, skin trouble, and scab.

8. The cosmetic composition according to claim 6, further comprising one or more components effective for skin conditions caused by sebum secretion.

9. The cosmetic composition according to claim 6, wherein the composition has a formulation selected from the group consisting of solutions, suspensions, emulsions, pastes, gels, creams, powders, soaps, cleansing containing surfactant, oils and sprays.

10. A method of inhibiting sebum secretion in a non-human animal, comprising a step of administering a compound represented by Formula 1 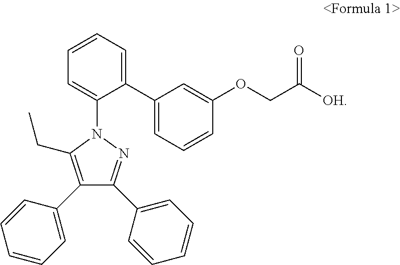 [SMILES: CCc1c(-c2ccccc2)c(-c2ccccc2)nn1-c1ccccc1-c1cccc(OCC(=O)O)c1]
